(19) United States
(12) Patent Application Publication
(10) Pub. No.: US 2011/0257655 A1
(43) Pub. Date: Oct. 20, 2011

(54) INSTRUMENT FOR MEASURING THE DISTRACTION PRESSURE BETWEEN VERTEBRAL BODIES

(76) Inventor: Franz Copf, JR., Stuttgart (DE)

(21) Appl. No.: 13/122,194

(22) PCT Filed: Oct. 2, 2009
(86) PCT No.: PCT/EP09/07085

§ 371 Date: Jun. 27, 2011

(30) Foreign Application Priority Data
Oct. 2, 2008 (DE) 10 2008 050 233.2

(51) Int. Cl. A61B 17/60; A61B 17/56

(52) U.S. Cl. 606/90; 606/102

(57) ABSTRACT

An instrument for measuring the distraction pressure between vertebral bodies comprises a control device (34, 36; 64, 66) for introducing a pressure force onto surfaces of opposing vertebral bodies and a measuring device (50; 84) associated with the control device (34, 36; 64, 66) for determining the pressure force applied onto the vertebral bodies. According to the invention, at least one end region of the control device (34, 36; 64, 66) carries a support body (10), which is designed for engaging in a concave dome that is surrounded by a bone ring of the vertebral bodies. The support body can be pivoted about at least one pivoting axis with respect to the control device and/or has a convex support surface, so that the support body when engaging into the dome can be pivoted in a sliding manner with respect to the vertebral body about at least one pivoting axis.

### BACKGROUND OF THE INVENTION

1. Field of the Invention

The invention relates to an instrument for measuring the distraction pressure between vertebral bodies, said instrument exhibiting an adjusting device for introducing a compressive force onto surfaces of opposed vertebral bodies and a measuring device, assigned to the adjusting device, for determining the compressive force exerted on the vertebral bodies.

An instrument of such a type is known from DE 603 01 238 T2.

2. Description of the State of the Art

One possibility for treating damage to intervertebral discs consists in removing the damaged intervertebral disc and replacing it with an intervertebral-disc prosthesis. The intervertebral-disc prosthesis is inserted into an intervertebral-disc compartment in the vertebral column, said compartment being delimited by two adjacently arranged vertebral bodies.

Intervertebral-disc prostheses are intended to guarantee as slight an impairment as possible of the mobility of the patient and are frequently constructed in the form of prosthetic plates which are connected to one another in articulated manner. In order to enable an advantageous adaptation of the intervertebral-disc prosthesis to the given anatomical conditions, from WO 2007/003438 A2 it is known to make intervertebral-disc prostheses available in a variety of configurations. The configurations differ, for example, with regard to the size of the prosthetic plates, the spacing between the prosthetic plates, and the position of the centre of motion, i.e. the fulcrum about which the prosthetic plates can be swivelled relative to one another. For this purpose the intervertebral-disc prosthesis exhibits intermediate elements which may have been constructed as joint elements or swivel-angle limiters. In the known prosthetic plates there are provided receiving shafts which are designed for receiving the intermediate elements. With the exchangeable intermediate elements the spacing and the maximal swivel angle between the prosthetic plates can be set and the position of the centre of motion can be established.

From WO 2007/003439 A2 an implantation procedure for an intervertebral-disc prosthesis is known wherein firstly the defective intervertebral disc is removed from the intervertebral-disc compartment between the vertebral bodies. Subsequently osseous hypertrophies are resected in such a manner that flat abutment surfaces for the prosthetic plates are created. Then, depending on the size of the intervertebral-disc compartment and on other given anatomical conditions, the configuration of the intervertebral-disc prosthesis to be inserted is established. For this purpose, suitable components—for instance, prosthetic plates, intermediate elements or wedge elements, which are arranged between the prosthetic plates and the abutment surfaces—are selected and assembled from sets of similar components.

In the case of a healthy vertebral column the vertebral bodies exert compressive forces on the intervertebral discs situated between them even when the vertebral column is not loaded. The compressive forces are generated, above all, by the ligamentous apparatus surrounding the vertebral column. With a view to avoiding faulty loadings, irritations and pathological changes in respect of the vertebral column, the inventor has perceived that these compressive forces should be preserved after the insertion of the intervertebral-disc prosthesis. For this purpose it is necessary to ascertain the compressive forces that are acting on the intervertebral discs in the case of a healthy vertebral column. This can be done, for example, with the aid of kinematic simulations of the vertebral column of the patient. If the ideal compressive forces are known, the size of the intervertebral-disc prosthesis—to be specific, in particular the spacing between the outer faces of the prosthetic plates—must be chosen in such a way that the ideal compressive forces arise. Corresponding remarks apply also to other types of intervertebral-disc implants, for example to cages and other fusing implants.

From DE 603 01 238 T2, mentioned in the introduction, a forceps-type distraction-pressure measuring instrument is evident which exhibits a load cell, arranged between actuating grips, for ascertaining a compressive force on the vertebral bodies, as well as a distance-measuring device. As soon as the treating physician has, with the aid of the known instrument, pressed the vertebral bodies sufficiently far apart, which he/she is able to judge on the basis of the compressive force ascertained by the load cell, the spacing of the vertebral bodies is read off on the distance-measuring device.

A disadvantageous aspect in this case, however, is that, owing to the shape of the instrument, an introduction of force during the distraction procedure occurs only on a small region of the hard osseous ring of the vertebral bodies. Consequently a tilting of the vertebral bodies cannot be ruled out, even with parallel guidance of the adjusting arms of the instrument. The distraction pressure is then measured under conditions such as do not generally prevail after the insertion of the intervertebral-disc prosthesis. A distraction pressure measured in this way is therefore not very meaningful and is only helpful in limited manner in connection with the configuration of the intervertebral-disc prosthesis.

### SUMMARY OF THE INVENTION

The object of the invention consists in improving an instrument of the type mentioned in the introduction in such a manner that the treating physician is able to measure the distraction pressure under more realistic and better reproducible conditions. This may in turn, for example, enable the physician to configure better the intervertebral-disc prosthesis to be inserted.

This object is achieved by an instrument with the features of Claim 1.

In accordance with the invention at least one end region of the adjusting device carries an abutment body which is designed for engagement in a concavely vaulted dome of the vertebral bodies which is enclosed by an osseous ring. The abutment body is capable of swivelling about at least one swivel axis in relation to the adjusting device and/or has an engagement region that is concavely vaulted in such a way that upon engagement in the dome the abutment body is capable of swivelling about at least one swivel axis by sliding motion in relation to the vertebral body.

With the aid of the instrument according to the invention the distraction forces are consequently introduced onto the vertebral body not exclusively on the osseous ring but also into the dome of the vertebral bodies which is enclosed by the osseous ring. The forces introduced into the dome cause the abutment body to bring itself, on its own, into a defined position in which it abuts the osseous ring not only in localised manner but over a circumferentially larger part. As a result, a situation is avoided in which the vertebral bodies are tilted too much when the distraction forces are introduced and therefore in which a pressure is measured that generally does not arise under real conditions.

In accordance with the invention, in the course of the measurement the abutment body can be swivelled in relation to the adjusting device about at least one swivel axis, but preferentially about three orthogonal swivel axes, in order that it can orient itself correctly as a consequence of the forces introduced into the dome. Such a swivelling capability can either be obtained by the abutment body firmly abutting the dome with its abutment surface but being arranged so as to be capable of tilting in relation to the adjusting device. Particularly when the vaulting of the abutment surface is at least substantially complementary to the concave vaulting of the dome, the abutment body is able to rotate independently in relation to the vertebral body into a particular orientation in which the abutment surface of the abutment body abuts the dome with maximal area contact. As a result, a defined orientation of the abutment body is created, which leads to better comparable results of measurement.

Generally a complementary vaulting will have the result that the abutment surface has at least substantially the shape a ramp. Particulars relating to this shape are gather from WO 2007/003438 A2, mentioned in the introduction (cf. in particular FIGS. 31 to 37), the content of which in this respect is made the subject-matter of the present application.

But a capability for relative swivelling between the adjusting device, on the one hand, and the vertebral body, on the other hand, can alternatively also be created by the abutment body having such a convexly vaulted abutment surface that upon engagement in the dome the abutment body is capable of swivelling about a swivel axis, preferentially about three swivel axes, by sliding motion in relation to the vertebral body. In the simplest case it is then a question, in the case of the abutment body, of a head with a spherical-cap-shaped abutment surface, which centres itself in the cupola of the dome on its own, so that as a result a defined position of the abutment body in relation to the vertebral body is also obtained. But the abutment body may also be designed in such a way that the vaulting of the abutment surface is at least substantially complementary to the vaulting of the dome. In this case the physician has to feel, by movements back and forth, the position in which the abutment surface of the abutment body has the greatest possible contact with the dome. In general, this will have the result that the abutment surface has at least substantially the shape a ramp.

Above all when the vaulting of the abutment surface is at least substantially complementary to the vaulting of the dome, which, as mentioned above, generally results in the formation of a ramp shape, the abutment body may be detachably connected to the adjusting device via a connecting device. This makes it possible to connect a variety of abutment bodies to the adjusting device. When choosing the abutment body to be connected to the adjusting device the physician may, for example, take into account biometric data that he/she has previously ascertained from the vertebral-column section in question of the patient to be treated.

Furthermore, the detachable connection between abutment bodies and adjusting device makes it possible to use abutment bodies that has at least partly the dimensions of a prosthetic plate to be inserted between the vertebral bodies. The prosthetic plates may have been adapted to the individual given anatomical conditions of the patient. The abutment body is then a type of ‘dummy prosthetic plate’ which, for example, exhibits a thickness that is reduced in relation to the prosthetic plate to be inserted later but otherwise (in particular with regard to the abutment surface) has the same shape.

Given a suitably designed connecting device, the possibility can even be created to use the prosthetic plate to be inserted later between the vertebral bodies by way of abutment body.

The connecting device may have been set up for a positive reception, in particular for a latching, of the abutment body. If, by way of abutment body, use is made of a prosthetic plate that has a receiving shaft for the purpose of fastening joint elements or swivel-angle limiters, the connecting device may have been set up for an engagement in such a receiving shaft. A dual function is then ascribed to the receiving shaft. Firstly, it serves for attaching the prosthetic plate to the instrument. After implementation of the measurement it serves for receiving the stated intermediate elements.

If the prosthetic plates are configured in such a way that their outer surfaces have the same spacings as the intervertebral-disc prosthesis to be inserted later, with the aid of the instrument it becomes possible to measure, in advance to a certain extent, the compressive forces acting on the intervertebral-disc prosthesis to be inserted. If the pressures measured in this process are too great or too small, the configuration of the intervertebral-disc prosthesis can still be modified by use being made, for example, of other intermediate elements, other prosthetic plates and/or other wedge elements.

In a further development of the invention there is provision that the connecting device has been set up for a positive reception, in particular for a latching, of the abutment body. A positive connection between adjusting device and abutment body guarantees safe handling of the instrument during the pressure-measuring procedure, which is advantageous in view of the size of the surgical opening which is to be kept as small as possible. For the positive connection an engagement of the end region of the adjusting device in an undercut region of the abutment body may have been provided.

The adjusting device preferentially includes a lever arrangement which has been set up for a manual introduction of force onto the end regions. By virtue of a forceps-type structure of the adjusting device, which is provided for a manual introduction of force by the user for the purpose of implementing the distraction procedure, a simple mechanical design of the instrument is made possible.

In a further development of the invention there is provision that the lever arrangement exhibits two arms that are relatively mobile with respect to one another at least substantially in parallel orientation. By this means, the tendency of the vertebral bodies to tilt during the implementation of the procedure for measuring the compressive force is reduced.

The instrument preferentially exhibits a distance-measuring device for determining the spacing between the vertebral bodies. The spacing measurement can also be implemented indirectly via other measured quantities. Although, when the instrument is locked in position after the distraction and after measuring the force and is drawn out of the intervertebral-disc compartment, this spacing can also be read off with the aid of a ruler or a similar measuring instrument placed against the abutment bodies, this is, on the one hand, error-prone on account of the generally curved outer vaulting of the abutment bodies and, on the other hand, comparatively time-consuming.

In a refinement of the invention there is provision that the adjusting device includes an adjusting element that is capable of being actuated by extraneous force, in particular a fluid cylinder. With the aid of the adjusting element it is possible to introduce the compressive force onto the vertebral bodies without a user having to exert considerable forces on the instrument. In addition, the compressive force can be built up in accordance with a predeterminable, in particular linear, characteristic. A compressive-force limitation may be provided, in order to prevent an exceeding of a predeterminable maximum pressure. The adjusting element is preferentially constructed as a fluid cylinder, into which a pressurised fluid, in particular a liquid, is introduced. Means for generating the extraneous force, for example a pump, may be either fitted directly to the instrument or accommodated in an external device.

In the case of the adjusting element it may be a question of a balloon that is capable of being filled with fluid, whereby changes in the volume of the balloon bring about a displacement of the abutment bodies. In comparison with a fluid cylinder such a balloon simplifies the structure of the instrument, since, for example, no seals for movable parts of the fluid cylinder are required.

In addition, a balloon, if it has an approximately spherical shape, can form a type of ball joint together with the abutment bodies. For this purpose it is expedient if the abutment bodies exhibit, on their insides facing towards the balloon, concave recesses, the surfaces of which form abutment surfaces for the balloon. The abutment bodies can then execute swivelling movements relative to the balloon and orient themselves optimally in the intervertebral-disc compartment before and also during the distraction. The degrees of freedom of the movement are even greater still in this case in comparison with a rigid joint.

When an adjusting element that is capable of being actuated by extraneous force is provided, the instrument may exhibit an interface to an external evaluating and controlling device. The interface may be designed in such a manner that the adjusting element is capable of being controlled from the external evaluating and controlling device. In this case it is not even necessary that the surgeon predetermines the requisite distractions himself/herself. Rather, this presetting can be effected by the evaluating and controlling device. If the evaluating and controlling device predetermines, for example, a sequence of distractions to be set, and the compressive forces arising in the process are measured, then the functional dependence between the spacing of the adjacent vertebral bodies and the distraction pressure prevailing between them can be derived from these pairs of values. This information can be helpful for the surgeon in connection with the establishment of the type and design of the implant to be inserted.

Via the interface, control commands and/or energy, in particular fluidic energy (i.e. fluid subject to overpressure or underpressure) or electrical energy, can be supplied to the adjusting element from the evaluating and controlling device. If the instrument itself has no energy storage device, a supply of energy via the interface will be required. Otherwise it is sufficient if nothing but control commands are supplied to the adjusting element via the interface.

In further refinement of the invention there is provision that the measuring device includes a pressure sensor and/or flexion sensor and/or torsion sensor. Hence a compressive force arising during the insertion and introduced onto the vertebral bodies via the instrument is ascertained. A pressure sensor enables a direct ascertainment of compressive force if it is arranged, for example, directly underneath the prosthetic plate on the instrument. With the aid of a flexion sensor or torsion sensor, deformations can be ascertained that are caused by reaction forces on the instrument.

The measuring device preferentially includes a display device which has been set up for outputting the result of measurement. The display device can output a measured value, in particular the measured compressive force, or a status signal that gives information about a settable compressive-force value being fallen short of, adhered to or exceeded. The measured values ascertained by the measuring device are preferentially made available to an evaluating device which has been set up for the calculation and display of the compressive forces arising and, where appropriate, for a provision of information that is helpful in connection with the selection of a suitable intervertebral-disc prosthesis.

The measuring device has preferentially been set up for a wireless transmission of the result of measurement to an evaluating device. As a result, the ease of handling of the instrument is enhanced, since no cable connection is necessary for transmitting the measured values ascertained by the measuring device. In particularly preferred manner the measuring device is designed in the manner of an RFID tag (radio-frequency identification technology transmission device) which, in particular without its own power supply, ascertains a measured value by excitation via an external electromagnetic field and makes it available to the evaluating device in wireless manner.

The invention further provides a system for measuring the functional dependence between the spacing between adjacent vertebral bodies and the distraction pressure prevailing between these vertebral bodies. Such a system exhibits an instrument for measuring the distraction pressure, which may be constructed in accordance with the invention but does not necessarily have to be. What is required is merely that the instrument exhibits an adjusting element which is capable of being actuated by extraneous force and with which a compressive force can be generated on surfaces of opposed vertebral bodies. The system further includes an evaluating and controlling device which is programmed in such a manner that it drives the adjusting element which is capable of being actuated by extraneous force in such a way that the vertebral bodies are distracted stepwise or continuously, as a result of which the spacing thereof increases. The programming further ensures that in the case of several different spacings the compressive force exerted between the vertebral bodies is determined and assigned to the respective spacing.

Such a system enables a largely automated determination of the functional dependence between the spacing of the vertebral bodies and the distraction pressure prevailing between them. With the aid of such a system this functional dependence can be determined within a few seconds, whereas a comparable manual measurement requires several minutes and, in addition, is error-prone.

A process according to the invention for configuring an intervertebral-disc prosthesis that is provided for implantation in an intervertebral-disc compartment formed between two opposed vertebral bodies exhibits the following steps:

a) inserting the abutment bodies of an instrument according to the invention into the domes of two vertebral bodies delimiting an intervertebral-disc compartment,

b) introducing a compressive force onto the vertebral bodies with the aid of the instrument until a predeterminable maximum pressure on the vertebral bodies and/or a predeterminable spacing between the vertebral bodies has/have been obtained,

c) measuring the pressure prevailing between the vertebral bodies and measuring the spacing between the vertebral bodies;

d) assembling an intervertebral-disc prosthesis from components, whereby at least one component is selected from a set of components that are similar to one another but that differ with regard to their dimensions or other properties by taking into consideration the quantities measured in step c).

### BRIEF DESCRIPTION OF THE DRAWINGS

Further features and advantages of the invention will become clear from the following description of an exemplary embodiment on the basis of the drawings. Shown therein are:

FIG. 1 a top view of a prosthetic plate,

FIG. 2 a sectional representation of the prosthetic plate according to FIG. 1,

FIG. 3 a view from below of the prosthetic plate according to FIG. 1,

FIG. 4 a first exemplary embodiment of an instrument according to the invention in a side view,

FIG. 5 the instrument according to FIG. 4 in a top view,

FIG. 6 a side view of another exemplary embodiment of an instrument in a neutral position, and

FIG. 7 a side view of the instrument according to FIG. 6 in an operating position

FIG. 8 a side view of another exemplary embodiment of an instrument in a neutral position, and

FIG. 9 a side view of the instrument according to FIG. 8 in an operating position.

### DESCRIPTION OF PREFERRED EXEMPLARY EMBODIMENTS

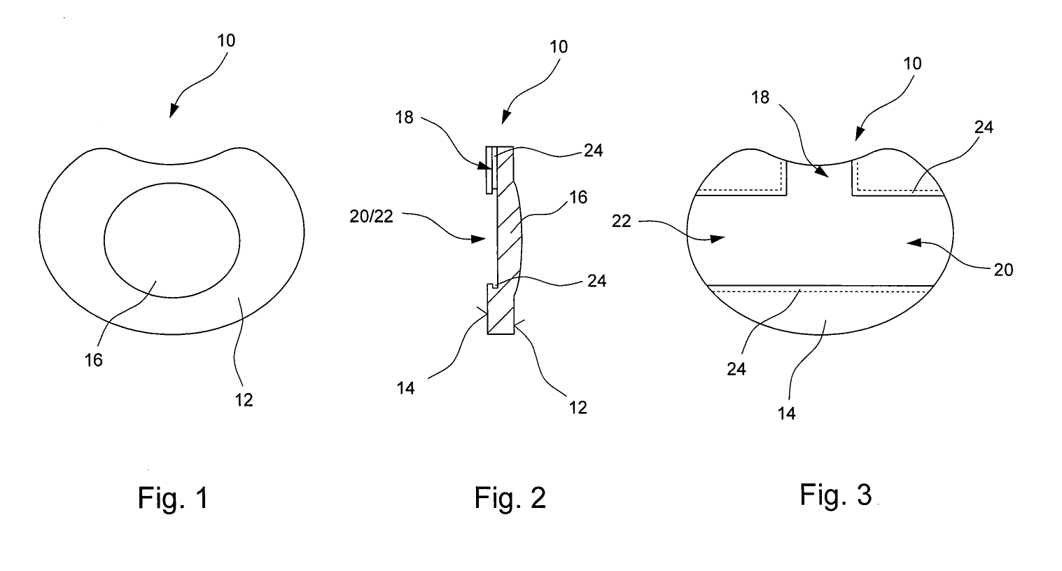

A prosthetic plate 10 represented in FIGS. 1 to 3 has been produced from a metallic material and exhibits a kidney-shaped outer contour which is evident in FIGS. 1 and 3. An upper side 12 of the prosthetic plate 10 serves for abutment against a vertebral body, which is not represented, and is provided with a vaulting 16 which is able to engage in a ring of harder bone material of the adjacent vertebral body. An underside 14 of the prosthetic plate 10 exhibits three shafts 18, 20, 22 which have been set up for receiving intermediate elements which are not represented. Shaft 18 serves for receiving a joint element which exhibits a concavely or convexly shaped spherical-cap-shaped region. The respective joint element forms with a corresponding joint element, which is attached to an opposite prosthetic plate 10, a ball joint with three rotational degrees of freedom of movement. Shafts 20 and 22 serve for receiving intermediate elements which are employed by way of swivel-angle limitation for the intervertebral-disc prosthesis to be formed from two oppositely arranged prosthetic plates 10. In order to guarantee a reliable reception of the intermediate elements in the shafts 18, 20, 22, in each instance in marginal regions of the shafts 18, 20, 22 there are provided grooves 24 which exhibit a rectangular cross-section and which enable a fixing of the intermediate elements in the manner of a tongue-and-groove joint. The cross-section of the groove 24 can be discerned well in the representation shown in FIG. 2. Further particulars relating to the prosthetic plate can be gathered from WO 2007/003438 A2, already mentioned in the introduction.

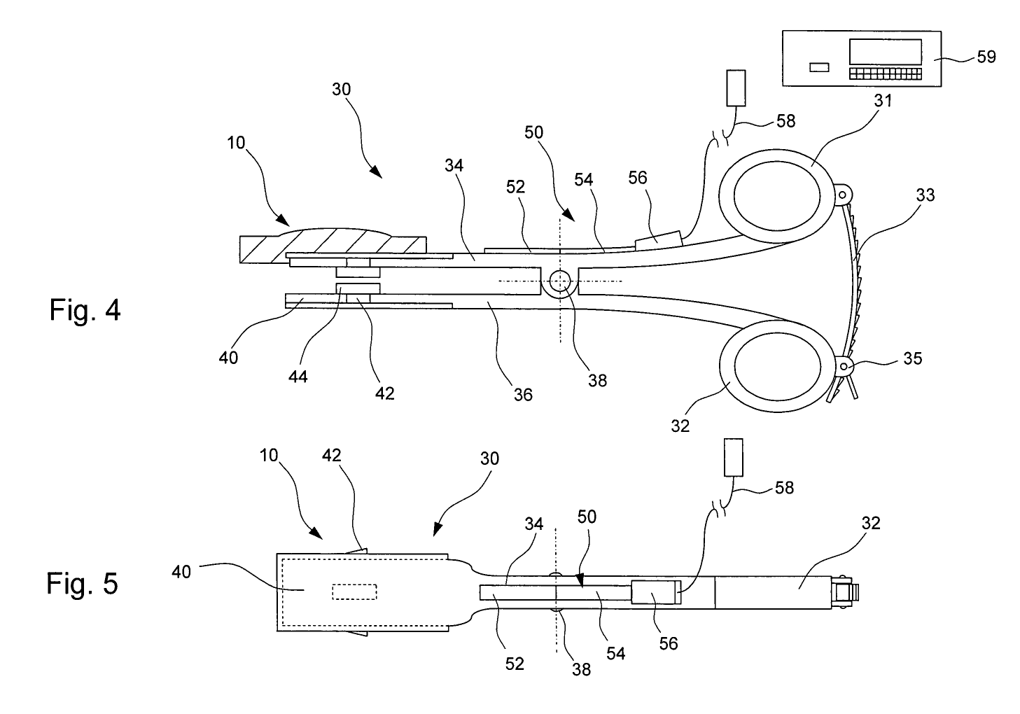

FIGS. 4 and 5 show an instrument 30 according to a first exemplary embodiment in a view from below and in a side view, respectively. The instrument 30 is constructed in the manner of a spreading instrument wherein annular gripping parts 31, 32 are attached terminally to ribs 34, 36. The ribs 34, 36 are connected to one another by means of a rivet 38 so as to be capable of swivelling in such a manner that a diminution of the spacing of the gripping parts 31, 32 results in an enlargement of the spacing between plate-carriers 40, widened in the form of a spade, of the instrument 30. On gripping part 31 there is attached in swivelling manner a curved rack 33 which is guided in a locking-pawl arrangement 35 on the opposite gripping part 32. The rack 33 is provided with a scale which is not represented and which enables the ascertainment of the spacing of the two gripping parts 31, 32 and hence indirectly the ascertainment of the spacing between the vertebral bodies.

In a cross-sectional plane oriented perpendicularly to the plane of the drawing of FIGS. 4 and 5 the plate-carriers 40 exhibit a T-shaped cross-section adapted to the profiling of the shafts 18, 20, 22 in the prosthetic plate 10. As a result, the prosthetic plate 10 can be pushed onto the respective plate-carrier 40 in positive manner in three different configurations. In FIG. 4 it is represented how the prosthetic plate 10, represented in sectional manner, has been pushed along the shaft 18 onto the plate-carrier 40.

In order to guarantee a reliable fixing of the prosthetic plate 10 to the instrument 30, on the plate-carriers 40 there project laterally spring-loaded barbs 42 which are each able to lock into the shafts 18, 20, 22 of the prosthetic plate 10. For the purpose of releasing the prosthetic plate 10 from the plate-carriers 40, in each instance slides 44 are provided which enable a movement of the barbs 42 out of the latching position represented in FIG. 4 into a neutral position, not represented, terminating flush with the outer edges of the plate-carrier 40.

On an upper side of rib 34 a measuring device 50 is provided which comprises two strain gauges 52, 54 arranged in series and a driver circuit 56. The strain gauges 52, 54 are firmly connected to the upper side of rib 34. Upon introduction of compressive forces onto the prosthetic plates 10 via the ribs 34, 36 a flexural deformation of the ribs 34, 36 takes place, as a result of which an elongation of the upper side of rib 34 occurs. This elongation results in a change of impedance in the strain gauges 52, 54. The change of impedance is a measure of the compressive force introduced into the ribs 34, 36 and acting on the vertebral bodies via the prosthetic plates 10. Measurement signals of the strain gauges 52, 54 are amplified in the driver circuit 56 and transmitted via a connecting cable 58 to an evaluating device 59 which is not represented. Therein the calculation of the compressive force and, incorporating the calculated compressive force and the spacing of the gripping parts 31, 32 read off on the rack, the ascertainment of the size of the intermediate elements to be employed for the intervertebral-disc prosthesis take place.

In an embodiment of an instrument which is not represented, the driver circuit for the strain gauges takes the form of an RFID tag and can, when a sufficiently strong electromagnetic field is present—such as can be made available, for example, by a brief transmitted pulse of the evaluating device—perform a query of the measured values of the strain gauges and subsequently transmit the ascertained measured values to the evaluating device in wireless manner.

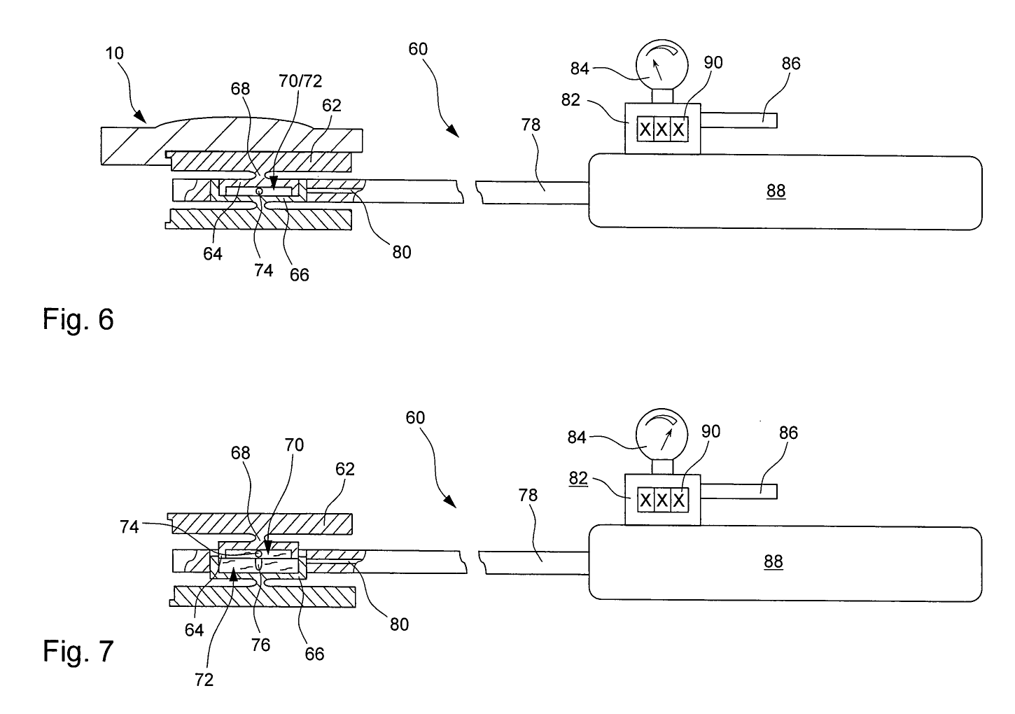

The exemplary embodiment of an instrument 60 which is represented in FIGS. 6 and 7 exhibits a hydraulic adjustment of the plate-carriers 62. The plate-carriers 62 are attached to pistons 64, 66 in swivelling manner via solid joints 68. The pistons 64, 66 are arranged nested within one another and each exhibit a circular cylindrical cross-section. By virtue of the nesting of the pistons 64, 66, with a very compact style of construction a sufficient piston stroke can be guaranteed which is necessary for the implementation of the ascertainment of compressive force.

The pistons 64, 66, each provided with cylindrical recesses 70, 72, delimit a working space into which a pressurised liquid can be introduced through lateral openings 74, 76, in order to press the pistons 64, 66 apart in opposite directions. Hence the force-transmitting abutment of the prosthetic plates 10 received on the plate-carriers 62 can be brought about on opposed vertebral bodies which are not represented.

The supply of the liquid is undertaken via a supply bore 80 which is provided in a guide rod 78. The supply bore 80 is in communication with a pump 82 to be actuated manually, which is arranged on a handle 88. Assigned to the pump 82 is a manometer 84 for indicating the liquid pressure prevailing in the working space between the pistons 64, 66. With the aid of an operating lever 86 attached to the pump 82 the pressure in the working space can be increased for such time until a predeterminable target pressure is indicated on the manometer 84. By means of a counter 90 assigned to the pump 82, on the basis of the number of pump strokes necessary for attaining the target pressure it can be ascertained at which spacing the prosthetic plates 10 have come to be situated, in order subsequently, based on the ascertained spacing, to make the selection of the intermediate elements.

In a variant which is not represented, the plate-carriers 62 do not carry any prosthetic plates but themselves form abutment bodies which come to abut the concavely vaulted dome of the vertebral bodies. The plate-carriers 62 may for this purpose exhibit a similar shape to that of the prosthetic plates 10. The plate-carriers may, however, also exhibit the shape of a spherical head.

Corresponding remarks also apply to the exemplary embodiment represented in FIGS. 4 and 5, in which the plate-carriers 40 are not capable of swivelling relative to the ribs 34, 36. A swivelling capability of the ribs 34, 36 in relation to the vertebral body is then obtained by virtue of the fact that the spherical head engaging in the dome of the vertebral body is able to rotate about two axes by sliding motion in relation to the vertebral body.

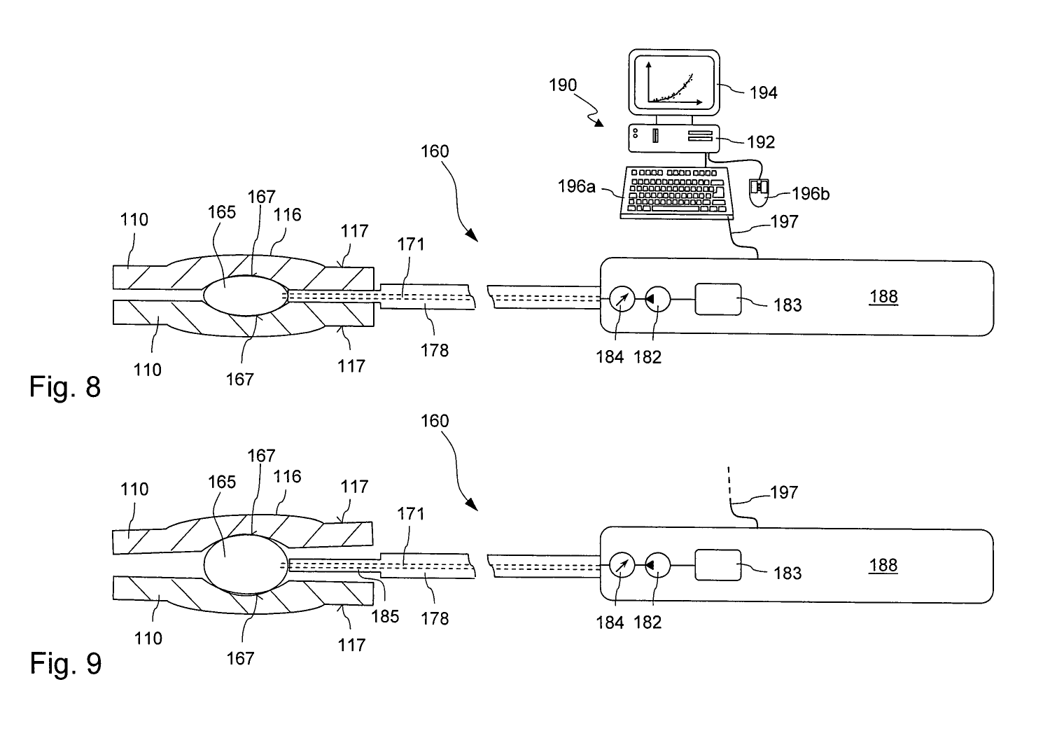

The exemplary embodiment of an instrument 160 represented in FIGS. 8 and 9 likewise exhibits a hydraulic adjustment. The working space, which in the exemplary embodiment represented in FIGS. 6 and 7 is delimited by the pistons 64 and 66, is here replaced by an elastic balloon 165, the volume of which is enlarged or can be reduced by supply or removal of liquid.

The instrument 160 further exhibits two abutment bodies 110 which on their sides pointing outwards to the vertebral bodies each exhibit a central convex vaulting 116 and an annular planar edge 117 surrounding the vaulting 116. The vaulting 116 is intended for engagement in a dome of the adjacent vertebral bodies, whereas the planar edge 117 comes to abut the osseous ring, surrounding the dome, of the vertebral bodies. The abutment bodies 110 are provided on their insides with concave recesses 167 which during the measurement jointly define a partially open cavity in which the balloon 165 is received. Both the outside of the balloon 165 and the concave recesses 167 of the abutment bodies are provided with a coatings lowering the friction, so that the abutment bodies are able to run off on the balloon 165 by sliding motion in a manner similar to that in the case of a ball joint.

The balloon 165 is connected to a pump 182 via a duct 171 and via a manometer 184. The duct 171 is received in a guide rod 178 which connects the balloon 165 to a handle 188. The handle 188 receives the manometer 184, the pump 182 and also a reservoir 183, fluidically connected thereto, for the liquid. The pump 182 is driven by an electric motor which is not represented and is able to pump a defined volume of the liquid from the reservoir 183 into the balloon 165 or back from the balloon 165 into the reservoir 183.

The abutment bodies 110 in this exemplary embodiment are not connected to the guide rod 178. In order to facilitate the insertion of the abutment bodies 110 into an intervertebral-disc compartment, a narrowed end portion 185 of the guide rod 178, which can be discerned well in FIG. 9, may consist of a permanent magnet or contain such a magnet. If the abutment bodies 110 consist of a paramagnetic material, the magnetic attraction forces suffice to connect the abutment bodies 110 in easily detachable manner to the guide rod 178, as is shown in FIG. 8. In this neutral position of the instrument the surgeon can comfortably introduce the abutment bodies 110, with the balloon 165 enclosed thereby, into the intervertebral-disc compartment.

The handle 188 of the instrument 160 is connected to an evaluating and controlling device 190 which exhibits, in addition to an arithmetical unit 192, a display device 194 and also input devices 196a, 196b. The evaluating and controlling device 190 controls the pump 182 via a control line 197. In the exemplary embodiment that is represented, the pump 182 is supplied directly with electrical energy via the control line 197. As an alternative to this, it is possible to receive in the handle 188 an energy storage device, for example in the form of a storage battery, which makes available the energy necessary for driving the pump 182. In this case, merely adjusting commands are communicated to the pump 182 via the control line 197. A further possibility to be considered is to receive also the manometer and the pump 182 in the evaluating and controlling unit 190 and to guide the duct 171 further, for example in the form of a flexible hose line, as far as the evaluating and controlling unit 190. The control line 197 or such a hose line represent an interface via which the controlling and evaluating device 190 can control the balloon 165.

The instrument represented in FIGS. 8 and 9 functions as follows:

Prior to the insertion of the abutment bodies 110 into the intervertebral-disc compartment the balloon 165 is emptied so far with the aid of the pump 182 that the abutment bodies 110 abut the narrowed end 185 of the guide rod 178, as shown in FIG. 8. Magnets which are present where appropriate hold the abutment bodies 110 in this neutral position.

Now the surgeon inserts the two abutment bodies 110, with the balloon 165 enclosed by them, into the intervertebral-disc compartment of the patient. After insertion has taken place, the surgeon acknowledges this by an input on one of the input devices 196a, 196b of the evaluating and controlling device 190. The further measuring procedure is now coordinated autonomously by the evaluating and controlling device 190; under certain circumstances it is merely necessary that the surgeon or the medical staff assisting the surgeon holds the handle 188 firmly during the measuring procedure.

During the measuring procedure the balloon 165 is gradually inflated with liquid by the pump 182, controlled by the evaluating and controlling device 190. The amount of the liquid volume added by the pump 182 to the balloon 165 in each instance is registered by the evaluating and controlling device 190. After each stepwise increase in the volume of the balloon 165 the manometer 184 measures the pressure of the liquid and passes it on via the control line 197 to the evaluating and controlling device 190. By virtue of the gradual inflation of the balloon 165 with liquid, the abutment bodies 110 abutting it are pressed apart against the resistance of the vertebral bodies abutting from outside, as a result of which the vertebral bodies are distracted. As a consequence of the sliding mounting of the abutment bodies 110 on the balloon 165, during the distraction said abutment bodies are able to execute swivelling movements in a manner similar to that in the case of a ball joint, as can be discerned in FIG. 9. In this way it is guaranteed that in the course of the distraction the flat edge 117 of the abutment bodies 110 always abuts, with its full surface or at least over a relatively large part of its periphery, the hard bone margin of the adjacent vertebral bodies.

Similarly as in the exemplary embodiment described with reference to FIGS. 6 and 7, also in the case of the instrument 160 the spacing between the adjacent vertebral bodies is determined indirectly from the volume of the balloon 165, from the geometry of the abutment bodies 110 and additionally by taking into consideration the pressure prevailing in the given case and measured by the manometer 184. The volume of the balloon 165 can easily be derived if the volume that is present in the balloon 165 at the start of the measurement is known and is then increased by the liquid volume that is added by the pump 182 during the measuring procedure. Consideration of the pressure prevailing in the balloon 165 and measured with the manometer 184 is sensible, since the shape of the balloon 165 and hence the spacing between the abutment bodies 110 changes as a function of the pressure. This pressure dependence of the shape of the balloon can be ascertained in simple manner by means of a calibration and can be saved in the evaluating and controlling device 190.

By this stepwise manner of proceeding, a plurality of pairs of values are obtained which each consist of the pressure measured by the manometer 184, on the one hand, and the spacing of the vertebral bodies which is determined from the volume of the balloon 165, taking the pressure into consideration. These pairs of values can be displayed on the display device 194 of the evaluating and controlling device 190 and, for example, can be supplemented by a fit curve. This functional dependence between the spacing of the adjacent vertebral bodies and the distraction pressure prevailing between these vertebral bodies can help the surgeon to select a suitable intervertebral-disc prosthesis or another implant.

Of course, this measuring procedure does not necessarily have to be implemented stepwise. In principle, a continuous measurement also enters into consideration.

However, care then has to be taken to ensure, by additional measures, that the pressure measurement by the manometer 184 is not falsified by the continuous pumping procedure.

1. Instrument for measuring the distraction pressure between vertebral bodies, having an adjusting device for introducing a compressive force onto surfaces of opposed vertebral bodies and a pressure-measuring device assigned to the adjusting device for determining the compressive force exerted on the vertebral bodies, wherein
at least one end region of the adjusting device carries an abutment body which is designed for engagement in a concavely vaulted dome of the vertebral bodies which is enclosed by an osseous ring, whereby the abutment body is capable of: a) swivelling about at least one swivel axis in relation to the adjusting device; or b) has such a convexly vaulted abutment surface-that upon engagement in the dome the abutment body is capable of swivelling about at least one swivel axis by sliding motion in relation to the vertebral body; or c) both a) and b).

2. Instrument according to claim 1, wherein the vaulting of the abutment surface is at least substantially complementary to the vaulting of the dome.

3. Instrument according to claim 1, wherein the abutment body is detachably connected to the adjusting device via a connecting device.

4. Instrument according to claim 3, wherein the abutment body has at least partially the dimensions of a prosthetic plate to be inserted between the vertebral bodies.

5. Instrument according to claim 4, wherein the abutment body is the prosthetic plate to be inserted between the vertebral bodies.

6. Instrument according to claim 3, wherein the connecting device has been set up for a positive reception of the abutment body.

7. Instrument according to claim 6, wherein the connecting device has been set up for an engagement in a receiving shaft provided in the abutment body.

8. Instrument according to claim 1, wherein the instrument includes a distance-measuring device for determining the spacing between the vertebral bodies.

9. Instrument according to claim 1, wherein the adjusting device includes an adjusting element that is capable of being actuated by extraneous force.

10. Instrument according to claim 9, wherein the adjusting element is a balloon that is capable of being filled with fluid, whereby changes in a volume of the balloon bring about a displacement of the abutment bodies.

11. Instrument according to claim 10, wherein, on their insides facing towards the balloon, the abutment bodies include concave recesses, the surfaces of which form abutment surfaces for the balloon.

12. Instrument according to claim 9, wherein the instrument includes an interface to an external evaluating and controlling device, the interface being configured so that the adjusting element is capable of being controlled from the external evaluating and controlling device.

13. Instrument according to claim 12, wherein control commands, energy, or both are capable of being supplied to the adjusting element via the interface.

14. Instrument according to claim 1, wherein the measuring device includes a display device which has been set up for outputting the result of measurement.

15. Instrument according to claim 1, wherein the measuring device has been set up for a wireless or wire-bound transmission of the result of measurement to an evaluating device.

16. System for measuring a functional dependence between a spacing of two adjacent vertebral bodies and the distraction pressure prevailing between the vertebral bodies, the system having:
an instrument according to claim 9, and
an evaluating and controlling device that has been programmed so that it
drives the adjusting element that is capable of being actuated by extraneous force in such a way that the vertebral bodies are distracted stepwise or continuously, as a result of which the spacing thereof is enlarged, and
in the case of several different spacings, determines the compressive force exerted between the vertebral bodies and assigns it to the respective spacing.

17. Set of abutment bodies for an instrument according to claim 1, wherein each abutment body exhibits a counter-connecting device, which is capable of being detachably connected to the connecting device of the instrument, and a convexly vaulted abutment surface, and wherein at least two abutment bodies of the set differ from one another in their shape, in their dimensions, or both.

18. Process for configuring an intervertebral-disc prosthesis which is provided for implantation into an intervertebral-disc compartment formed between the vertebral bodies, the process including the steps of:
a) inserting the abutment bodies of an instrument according to claim 1 into the domes of the vertebral bodies delimiting an intervertebral-disc compartment,
b) introducing the compressive force onto the vertebral bodies with the aid of the instrument until a predeterminable maximum pressure on the vertebral bodies, a predeterminable spacing between the vertebral bodies, or both, has been obtained,
c) measuring the pressure prevailing between the vertebral bodies and measuring the spacing between the vertebral bodies, and
d) assembling an intervertebral-disc prosthesis from components, whereby at least one component is selected from a set of components that are similar to one another but that differ with regard to their dimensions or other properties by taking into consideration the quantities measured in step c).

19. Instrument according to claim 2, wherein the abutment surface has at least substantially the shape of a ramp.

20. Instrument according to claim 6, wherein the connecting device has been set up for a latching of the abutment body.

21. Instrument according to claim 9, wherein the adjusting element is capable of being activated by a fluid cylinder.

22. Instrument according to claim 13, wherein the energy is fluidic or electrical energy.
